(19) United States
(12) Patent Application Publication
(10) Pub. No.: US 2012/0203270 A1
(43) Pub. Date: Aug. 9, 2012

(54) FLEXURAL LAPARASCOPIC GRASPER

(76) Inventors: Yuanyu Chen, Cambridge, MA (US); Audrey Paulette Solange Maertens, Canton, MA (US); Harrison Fagan O'Hanley, Ipswich, MA (US); Matthew Jon Rosario, Cambridge, MA (US); John Michael Walton, Meredith, NH (US); Mitchell Terrance Westwood, Boston, MA (US); Jennifer Erica Rosen, Wellesley, MA (US)

(21) Appl. No.: 13/367,033

(22) Filed: Feb. 6, 2012

Related U.S. Application Data
(60) Provisional application No. 61/439,612, filed on Feb. 4, 2011.
(60) Provisional application No. 61/439,649, filed on Feb. 4, 2011.
(60) Provisional application No. 61/484,305, filed on May 10, 2011.
(60) Provisional application No. 61/494,007, filed on Jun. 7, 2011.

(51) Int. Cl. A61B 17/29

(52) U.S. Cl. 606/205

(57) ABSTRACT

A flexural grasper for use in laparoscopic procedures and the like includes an end effector with digits having rigid elements coupled by flexural bearings. The flexural bearings can be controlled to flex and straighten using cables deployed through the digits and tensioned remotely from a handle for the flexural grasper.

### CROSS-REFERENCE TO RELATED APPLICATIONS

This application claims the benefit of U.S. Prov. App. No. 61/439,612 filed on Feb. 4, 2011, U.S. Prov. App. No. 61/439,649 filed on Feb. 4, 2011, U.S. Prov. App. No. 61/484,305 filed on May 10, 2011, and U.S. Prov. App. No. 61/494,007 filed on Jun. 7, 2011. Each of the foregoing applications is hereby incorporated by reference in its entirety.

### FEDERALLY SPONSORED RESEARCH

This invention was made with Government support under Grant No. W81XWH-09-2-0001 awarded by the U.S. Army Medical Research and Materiel Command. The Government has certain rights in this invention.

### BACKGROUND

There remains a need for improved surgical grasping tools for use in laparoscopic surgery or other similarly constrained procedures.

### SUMMARY

A flexural grasper for use in laparoscopic procedures and the like includes an end effector with digits having rigid elements coupled by flexural bearings. The flexural bearings can be controlled to flex and straighten using cables deployed through the digits and tensioned remotely from a handle for the flexural grasper.

### BRIEF DESCRIPTION OF THE FIGURES

The invention and the following detailed description of certain embodiments thereof may be understood by reference to the following figures.

FIG. 1 shows a flexural grasper.

FIG. 2 shows an end effector in an open configuration.

FIG. 3 shows a cross section of a collapsed three-digit end effector.

FIG. 4 shows a digit of an end effector.

FIG. 5 shows a digit of an end effector.

FIG. 6 shows a digit of an end effector.

FIG. 7 shows a handle for a flexural grasper.

### DETAILED DESCRIPTION

The following description details embodiments of a flexural grasper for use in laparoscopic or endoscopic surgery. It will be understood, however, that the principles of the invention may be usefully adapted to any environment where controlled grasping is required through a small port and all such uses and adaptations that would be apparent to one of ordinary skill in the art are intended to fall within the scope of this disclosure. It will further be appreciated that the following description provides details of various embodiments by way of example and not of limitation.

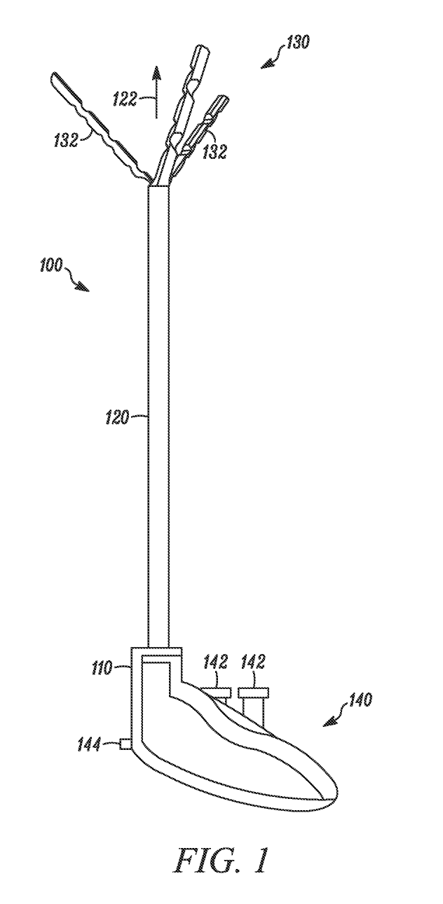

FIG. 1 shows a flexural grasper. In general, the flexural grasper 100 may include a handle 110, a transmission shaft 120, an end effector 130, and a trigger mechanism 140 within the handle 110.

The handle 110 may be generally shaped and sized to be held by a surgeon or other technician during a laparoscopic surgical procedure or the like. The handle 110 may be molded for hand gripping, and may include a pliable material, grip-enhancing material or any other suitable coating(s) or other materials to improve use of the flexural grasper 100.

The transmission shaft 120 may be shaped and sized to fit through a laparoscopic trocar port, or more generally any port through which controlled gripping is desired. This may for example, include an endoscopic port or any other surgical or non-surgical opening. The transmission shaft 120 may, for example, have a cross-sectional size and shape to fit within a twelve millimeter laparoscopic trocar port. The transmission shaft 120 may be generally linear in shape (consistent with a laparoscopic tool) with a central axis 122. It will be appreciated that the central axis 122 is described for reference only, and is not intended to imply any specific linearity or symmetry for the transmission shaft 120.

The end effector 130 may include a number of digits 132 or fingers. Each digit 132 may in turn include a number of rigid elements sequentially coupled by a number of flexural bearings, as described in greater detail below.

The trigger mechanism 140 may be generally configured to tension one or more cables for each one of the digits 132 in the end effector 130, thus applying a bending force to one or more of the number of flexural bearings as described below. The trigger mechanism 140 may include one or more triggers 142 and a release 144 to controllably tension the cable(s) (not shown) running through the transmission shaft 120 to the end effector 130, so that the flexural bearings can be controllably flexed, e.g., to grip an object.

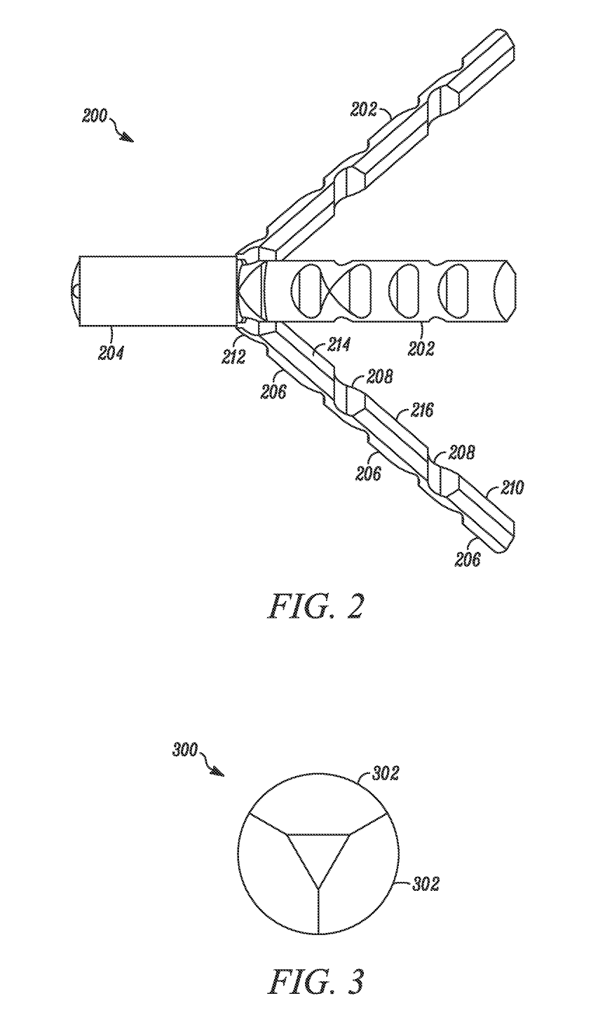

FIG. 2 shows an end effector in an open configuration. In general, the end effector 200 includes a number of digits 202 coupled to a base 204. It will be appreciated that the digits 202 and the base 204 may be formed of a single, integral piece of material, or one or more of the digits 202 may be single integral pieces each assembled into the base 204, or any number of pieces may be separately fabricated and assembled using any suitable techniques. As described below, a cable may be used to bend the flexural bearings of each digit 202. In one aspect, a first flexural bearing may have an angle to the central axis of the transmission shaft when at rest, so that the end effector 200, in the absence of applied forces, has an open configuration as depicted. It will be understood that, while three digits 202 are depicted in FIG. 2, any number of digits 202 may be suitable employed, including without limitation two digits 202, three digits 202, or four digits 202.

In general, each digit 202 may include a number of rigid elements 206 sequentially coupled by a number of flexural bearings 208. A cable (not shown) may extend from the handle of the flexural grasper described above through a transmission shaft to a terminal one 210 of the rigid elements. In one configuration, a first one of the flexural bearings 212 between an initial one of the rigid elements (within the base 204 and not visible in FIG. 2) and a second one of the rigid elements 214 has an angle to the central axis of the transmission shaft when at rest (e.g., not under any cable tension or other applied force). Each other one of the flexural bearings 208 may couple two of the rigid elements 206 collinearly when at rest, so that the digits 202 form a number of straight lengths angled away from each other and the central axis of the transmission shaft of the flexural grasper.

The base 204 may retain the digits 202 in a desired initial configuration, subject to control of the flexural bearings 208 as described below.

FIG. 3 shows a cross section of a collapsed three-digit end effector. When the digits 302 of the end effector 300 are collapsed together, such as when the initial flexural bearing for each digit 302 is bent to bring the digits 302 into collinear alignment (or more precisely, parallel to collinear) with the central axis of the transmission shaft, the end effector 300 may have a generally circular cross-section that can fit within, e.g., a corresponding circular cross section of a port of a laparoscopic tool or the like. In this manner, the end effector 300 may be retracted into an opening within the transmission shaft, and/or deployed from within the transmission shaft into an open position for use, thus permitting insertion and removal of the end effector into a surgical site or cavity through a narrow bore. As shown in FIG. 3, the digits 302 may be evenly spaced around the central axis of the transmission shaft, although this arrangement is not required.

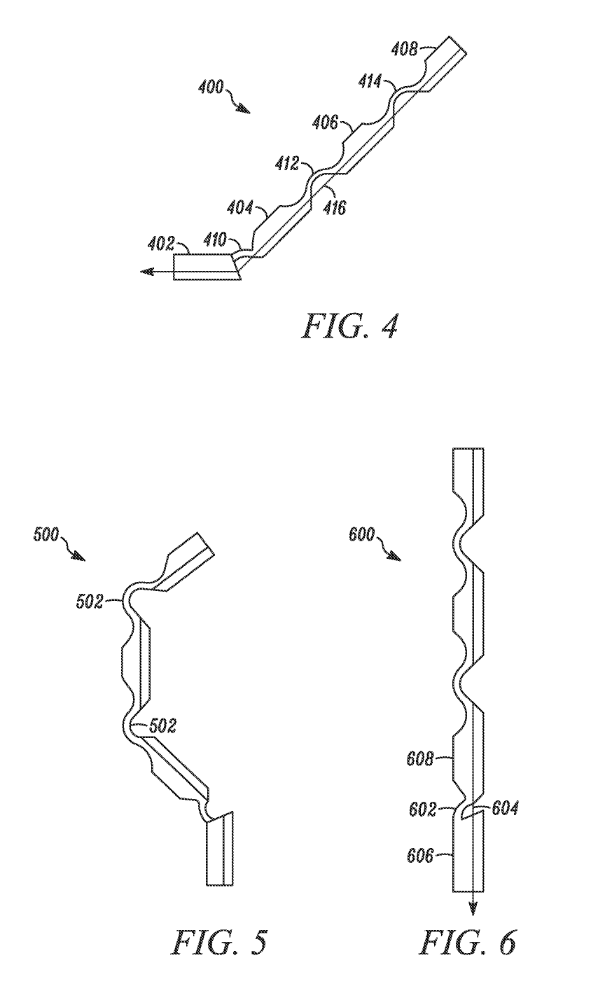

FIG. 4 shows a digit of an end effector. As described above, the digit 400 generally includes a number of rigid elements and a number of flexural bearings. More specifically, the digit 400 depicted in FIG. 4 includes a first rigid element 402 (the initial rigid element), a second rigid element 404, a third rigid element 406, and a fourth rigid element 408 (the terminal rigid element), coupled respectively by a first flexural bearing 410, a second flexural bearing 412, and a third flexural bearing 414. The digit 400 may be fabricated from a single, integral piece of material, or the digit 400 may be fabricated from separate parts for each rigid element and/or flexural bearing, or some combination of these.

A cable 416 may be attached to the terminal rigid element and may pass through the digit 400, and then through the transmission shaft of a flexural grasper, as indicated generally by an arrow. The digit 400 may include a channel or other opening or groove for the cable 416 to permit free movement except where attached to the terminal rigid element. When tension is applied to the cable 416, one or more of the flexural bearings may bend (according to relative stiffness) so that the digit 400 deforms into a curved shape or the like.

In one aspect, the digit may include four rigid elements interconnected by three flexural bearings as depicted in FIG. 4. However, it will be understood that a variety of arrangements are possible including more or less rigid elements and more or less flexural bearings. Furthermore, the flexural bearings may be designed for a variety of different shape responses to an applied force. For example, in one aspect each of the flexural bearings, or each of the flexural bearings that couples rigid elements collinearly when resting, may have an equal stiffness so that an applied force bends each flexural bearing equally. In another aspect, each flexural bearing may have a different stiffness selected to achieve any predetermined flexing pattern. So for example, it may be desirable to have the terminal flexural bearings flex more than other flexural bearings in order to surround a large object within a cavity. The terminal flexural bearings may be made correspondingly less stiff to achieve this flexing pattern. In another aspect, the stiffness may be equal among the flexural bearings in order to achieve a curling pattern as depicted for example in FIG. 6 below. In another aspect, the predetermined flexing pattern may fold progressively from the terminal flexural bearing distal to the transmission shaft to one of the flexural bearings proximate to the transmission shaft. More generally, the stiffness of the flexural bearings may be selected to achieve a variety of predetermined bending patterns.

FIG. 5 shows a digit of an end effector. As a force is applied to each of the flexural bearings 502 of the digit 500, such as with the cable described above, the flexural bearings may be urged to bend, thus creating a curved shape to the digit 500.

FIG. 6 shows a digit of an end effector. In this embodiment, the digit 600 includes a first flexural bearing 602 that has an angle when at rest, that is, without any apply force. With a cable 604 positioned between the initial rigid element 606 and a second rigid element 608 and securely attached to the second rigid element 608, the first flexural bearing may be urged into alignment with the remaining rigid elements of the digit 600 to effectively straighten the digit 600 along its entire length. In general, the cable 604 may be used instead of or in addition to the cable described above for greater control over the digit 600. 13. Thus in one aspect, the flexural grasper may include a second cable for each one of the digits that extends from the handle through the transmission shaft and the initial one of the rigid elements to the second one of the rigid elements, where the second cable is securely fastened. This second cable may apply a bias force to the first one of the flexural bearings so that it is urged from its initial, at rest orientation (angled to the central axis) toward an orientation collinear with the central axis.

In another aspect, the first flexural bearing 602 may be made substantially stiffer than other flexural bearings in order to prevent curling/grasping formations from collapsing the first bearing into collinear alignment with the transmission shaft. In another aspect, the second cable may be omitted entirely so that the first flexural bearing 602 only bends during refraction and deployment of the end effector, e.g., under the applied forces of an interior bore of the transmission shaft of the flexural grasper.

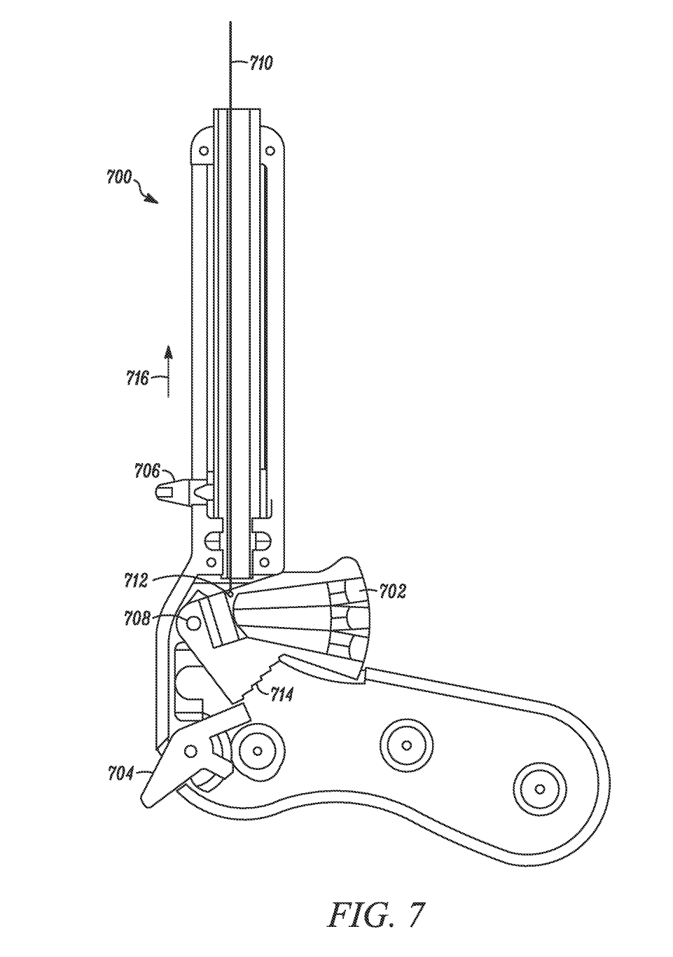

FIG. 7 shows a handle for a flexural grasper. In general, the handle 700 includes a triggering mechanism with a trigger 702 and a release 704, along with a lever 706 to deploy an end effector.

The trigger 702 may be positioned for finger actuation by a user gripping the handle 700, and may be rotatably coupled to a pivot point 708 within the handle 700. A cable 710 may be coupled to the trigger 702 at a connection point 712 so that, by applying finger pressure to the trigger 702, the trigger 702 rotates about the pivot point 708 to tension the cable 710. In this manner, the end effector (not shown) described above may be actuated to bend with a predetermined flexing pattern. It will be understood that while the cable 710 is depicted as a simple, linear element, the cable 710 may include any number of pulleys or other mechanical elements to achieve a desired mechanical advantage. By adjusting the mechanical arrangement of the cable 710, and suitably placing the pivot point 708 and the connection point 712, the trigger 702 may be configured to provide various ranges of motion and/or actuating forces for the digits of the end effector. It will also be understood that each digit may have a separate cable branching from the cable 710 coupled to the trigger 702, and that each such separate cable may be independently configured through suitable couplings for a different range of motion. Thus each digit may have a different range of motion according to the cable couplings, and may also have a different predetermined flexing pattern according to the stiffness of the flexural bearings. Using these characteristics, a wide range of gripping motions may be achieved. In other embodiments, a number of triggers may be provided so that, for example, each digit can be controlled independently, or different flexural bearings can be controlled independently. For example, a second cable may be coupled to a second trigger that provides movement of the digits of an end effector into a collapsed position so that they are all parallel to the central axis of a transmission shaft as described above.

The trigger 702 may include a ratchet 714 that engages a tooth on the release 704. When the trigger 702 is activated (e.g., rotated), the teeth of the ratchet may progressively engage the tooth on the release 704 to provide unidirectional cable tensioning of the cable 710. By activating the release 704, the tooth on the release 704 can rotate out of engagement with the ratchet 714 on the trigger 702 and release any tension on the cable 710, thus restoring the end effector to its relaxed position. A second ratchet, or any number of additional ratchets may be provided for unidirectional tensioning of additional cables, such as a second cable for collapsing the end effector, along with additional releases.

In another aspect, some or all of the cabling may be replaced with rigid rods. In one aspect, a rigid rod may be used in place of the cable 710 along the length of the transmission shaft in order to reduce loose cabling in the shaft when the cables in the end effector are not being tensioned. In another aspect, an entire cabling network from the trigger 702 to one or more of the terminal rigid elements (or intermediate rigid elements as appropriate) may be replaced with rigid rods for bidirectional translation of control forces. That is, with one or more rigid rods extending to the end of a controlled digit, flexural bearings may be tensioned (e.g., pulled inward by the trigger) or pressured (e.g., pushed out by the trigger) for direct control of opening and closing movements of the end effector. In another aspect, a spring or similar arrangement may be used within the trigger 702 to bias the trigger 702 in any desired direction, such as toward an open position where the end effector is opened to prepare for grasping. More generally, a variety of arrangements of springs, rods, cables, and pre-biased flexural bearings, or any other mechanical mechanisms, either alone or in combination, may be employed to establish a desired equilibrium (i.e., resting) position of the end effector, and to permit movement to open or close the grasping digits under control of the trigger 702.

It will be understood that while the ratcheting trigger described above provides a simple, convenient mechanical framework for controlling cable tensioning to manipulate an end effector, a variety of other techniques of varying complexity are known in the art, and any such mechanisms suitable for controlling cable tension and/or position through a long narrow passage such as the transmission shaft may be suitably adapted to use with the flexural grasper contemplated herein.

The lever 706 may, for example, be a sliding lever coupled to the base of the end effector with a stiff rod or the like. In this configuration, a forward motion of the lever 706 (indicated generally by an arrow 716) can be translated into a movement of the base of the end effector so that the digits of the end effector extend out of the transmission shaft into a deployed position. A contrary motion can be employed to retract the digits of the end effector into the transmission shaft, which may have sufficient volume to receive the end effector in its collapsed form.

While particular embodiments of the present invention have been shown and described, it will be apparent to those skilled in the art that various changes and modifications in form and details may be made therein without departing from the spirit and scope of this disclosure and are intended to form a part of the invention as defined by the following claims, which are to be interpreted in the broadest sense allowable by law.

What is claimed is:

1. A device comprising:
a handle;
a transmission shaft shaped and sized to fit through a laparoscopic trocar port, the transmission shaft having a central axis;
an end effector including a number of digits, each one of the digits including a number of rigid elements sequentially coupled by a number of flexural bearings, a first cable extending from the handle through the transmission shaft to a terminal one of the rigid elements, wherein a first one of the flexural bearings between an initial one of the rigid elements and a second one of the rigid elements has an angle to the central axis when at rest, and each other one of the flexural bearings couples two of the rigid elements collinearly to one another when at rest; and
a trigger mechanism within the handle, the trigger mechanism configured to tension the first cable for each one of the digits, wherein the first cable applies a bending force to one or more of the flexural bearings that couples two of the rigid elements to one another.

2. The device of claim 1 wherein the digits includes three digits.

3. The device of claim 1 wherein the digits are spaced evenly about the central axis of the transmission shaft.

4. The device of claim 1 wherein each one of the digits includes four rigid elements interconnected by three flexural bearings.

5. The device of claim 1 wherein each one of the flexural bearings that couples two of the digits collinearly has an equal stiffness.

6. The device of claim 1 wherein each one of the flexural bearings that couples two of the digits collinearly has a stiffness selected for a predetermined flexing pattern of the end effector.

7. The device of claim 6 wherein the predetermined flexing pattern includes a curling pattern.

8. The device of claim 6 wherein the predetermined flexing pattern folds progressively from one of the flexural bearings distal to the transmission shaft to one of the flexural bearings proximate to the transmission shaft.

9. The device of claim 1 wherein each one of the digits is fabricated from a single, integral piece of deformable material with the flexural bearings formed therein.

10. The device of claim 1 wherein the triggering mechanism includes a trigger coupled to the first cable.

11. The device of claim 10 wherein the trigger includes a ratchet for unidirectional cable tensioning of the first cable.

12. The device of claim 11 wherein the trigger includes a release to release the unidirectional cable tensioning.

13. The device of claim 1 further comprising a second cable for each one of the digits extending from the handle through the transmission shaft and the initial one of the rigid elements to the second one of the rigid elements.

14. The device of claim 13 wherein the second cable applies a bias force to the first one of the flexural bearings from the angle to the central axis toward an orientation collinear with the central axis.

15. The device of claim 14 wherein the triggering mechanism includes a second trigger coupled to the second cable.

16. The device of claim 15 wherein the second trigger includes a second ratchet for unidirectional cable tensioning of the second cable.

17. The device of claim 16 wherein the second trigger includes a second release to release the unidirectional cable tensioning of the second cable.

18. The device of claim 1 wherein the shaft includes an opening on an end distal to the handle having a volume sufficient to receive the end effector.

19. The device of claim 18 wherein the handle includes a lever to move the end effector to a first position within the opening of the shaft to a second position outside the opening of the shaft.

20. The device of claim 1 wherein the transmission shaft is shaped and sized to fit through a twelve millimeter laparoscopic trocar port.
